(19) United States
(12) Patent Application Publication
(10) Pub. No.: US 2022/0273164 A1
(43) Pub. Date: Sep. 1, 2022

(54) SYSTEM AND METHOD FOR DISPLAYING IMAGES OF A MEDICAL IMAGING ENDOSCOPE

(71) Applicant: 270 SURGICAL LTD., NETANYA (IL)

(72) Inventors: MOSHE LEVI, GANEY TIKVA (IL); GOLAN SALMAN, ATLIT (IL); AVRAHAM LEVY, KFAR SHMARYAHU (IL)

(21) Appl. No.: 17/632,540

(22) PCT Filed: Jul. 13, 2020
(86) PCT No.: PCT/IL2020/050782

§ 371 Date: Feb. 3, 2022

Related U.S. Application Data
(60) Provisional application No. 62/882,618, filed on Aug. 5, 2019.

(51) Int. Cl. A61B 1/00; G06T 7/00

(52) U.S. Cl. CPC A61B 1/000095; A61B 1/0005; G06T 7/0012; A61B 1/00042; G06T 2207/10068

(57) ABSTRACT

The subject matter discloses a medical imaging system and method for generating a video configuration from multiple cameras of a medical imaging endoscope. The system comprises a processing unit, operatively connected to the endoscope and to a monitor, the processing unit is instructed to receive multiple video data streams captured from the multiple cameras located at a distal tip of the endoscope; receive from the system a video mode defining a layout of the multiple video data streams; perform a video interpolation on the multiple video data streams according to the video mode; generate a video configuration for the multiple video data streams, the video configuration characters each video data stream of the multiple video data streams in different properties in accordance with the received video mode; send the video configuration to the monitor, and display the video configuration layout on the monitor.

### FIELD OF THE INVENTION

The present invention relates generally to the field of medical instruments designed to capture images from inside a lumen.

### BACKGROUND OF THE INVENTION

Endoscopes are medical devices which have been utilized to perform operations in the internal organs of the body through small incision in skin. Over the years such medical devices have utilized to perform operations and capture images in internal organs of a body. Such medical devices have been developed and categorized according to specific applications, such as laparoscopes, arthroscopes, cystoscopes, ureteroscopes, hysterectomy and others. In some cases, these medical devices can enter the body through small incisions in the body. Typically, such operations require the aid of a camera and in some cases, more than one camera. As of today, there are multiple different types of flexible endoscopes, rigid and semi-rigid endoscopes, depending on the area/lumen in which the device is used and the procedure's type. As a result of that, to enable the required field of view during the medical procedure the scope has to be directed to the region of interest constantly.

A standard rigid and semi-rigid endoscope is likely to be designed as an elongated tubular member in which the optical gear is located at its distal tip, as well as all the electrical circuitry. In most cases, the flexible endoscopes are inserted into the patient's body via natural orifices, while rigid and semi-rigid endoscopes typically inserted into the body via small incisions, which can be about 11 millimetres or less, made in the patient's body.

### SUMMARY OF THE INVENTION

It is an object of the subject matter to disclose a medical imaging system for generating a video configuration from at least two cameras of a medical imaging endoscope, wherein the medical imaging system comprises a processing unit, wherein the processing unit is operatively connected to the medical imaging endoscope and to a monitor, and wherein the processing unit of the medical imaging system is instructed to receive at least two video data streams captured from the at least two cameras located at a distal tip of the medical imaging endoscope, receive from the medical imaging system a video mode defining a layout of the at least two video data streams, perform a video interpolation on each of the at least two video data streams according to the video mode, generate a video configuration for the at least two video data streams, wherein the video configuration characters each video data stream of the at least two video data streams in different size, scale, magnitude, frame rate and video display aspect ratio in accordance with the received video mode, send the video configuration to the monitor, and display the video configuration layout defined by the video mode of the at least two video data streams simultaneously on the monitor.

In some cases, the medical imaging system further comprising a control means configured to receive the video mode that dictates the layout of the video configuration. In some cases, the medical imaging system is further designed to modify the parameters defining the characterizations of the displays of the at least two video data streams for changing the layout of a displayed video configuration. In some cases, the video interpolation is performed independently on each of video data stream of the at least two video data streams.

In some cases, the video interpolation comprising utilizing parameters which define characteristics of the display, the parameters are selected from a list comprising a number of video frames to output, frame rate, timestamps assuming constant frame rate, video display aspect ratio, pixel format defining the memory layout of each pixel in a bitmap, overlap lines and magnitudes, such that the processing unit is further configured to receive data of the at least two video data streams, follow by perform the video interpolation according to a predefined video mode for obtaining the video configurations. In some cases, the video display aspect ratio is 4:3, 16:9, 16:10, 32:9.

It is another object of the subject matter to disclose a method for displaying a video configuration of at least two cameras of a medical imaging endoscope by a medical imaging system comprises a processing unit operatively connected with the medical imaging endoscope and with a monitor, the method comprising receiving at least two video data streams captured by at least two cameras located at the medical imaging endoscope in the processing unit, receiving from the medical imaging system a video mode defining a layout of the at least two video data streams, performing a video interpolation on each of the at least two video data streams based on the received video mode, generating a video configuration from the at least two video data streams, wherein the video configuration defines each video data stream of the at least two video data streams is different in one or more video characteristics, transmitting the video configuration to the monitor, and displaying the video configuration layout on the monitor, such that the at least two video data streams are displayed simultaneously.

In some cases, the video mode is received from control means of the medical imaging system. In some cases, the control means comprise at least one of buttons, switches, knobs placed within a handle of the medical imaging endoscope. The method of claim 8, wherein the control means comprise at least one of buttons, switches, knobs placed within the processing unit.

It is another object of the subject matter to disclose a method for displaying a layout of a video configuration based on video data streams captured by three cameras of a medical imaging device, comprising receiving three video data streams captured by the three cameras located within the medical imaging device, generating the video configuration from the three video data streams using a main control unit of the medical imaging device and based on a predefined video mode, said video configuration comprises four video displays, wherein three video displays of the four video displays relates to the video data streams captured by the three cameras and a fourth video display comprises a video data stream captured by a selected camera of the three cameras, wherein the four video displays are simultaneously displayed on a monitor.

In some cases, the fourth video display has a video characteristic different from the three video displays' video characteristic. In some cases, the video characteristic selected from a list comprising size, scale, magnitude, frame rate, video display aspect ratio and a combination thereof.

It is another object of the subject matter to disclose a method for displaying a video configuration based on video streams captured by three cameras of a medical imaging endoscope by a medical imaging system comprises a processing unit operatively connected with the medical imaging endoscope and with a monitor, the method comprising receiving three video data streams captured by the three cameras located within the medical imaging endoscope in the processing unit, receiving from the medical imaging system a video mode defining a layout of the three video data streams into four video displays, to be simultaneously displayed on the monitor, performing a video interpolation on each of the three video data streams based on the video mode, generating a video configuration based on the three video data streams, wherein the video configuration comprises three video displays having different video characteristics, each display of the three displays is provided from a different video stream of the characters each video data stream in different size, scale, magnitude, frame rate and video display aspect ratio and wherein the video mode comprises display of the three video data streams at the four video displays, setting parameters defining the characterizations of each of the four video displays of the three video data streams in accordance with the received video configuration, transmitting the video configuration to the monitor, and displaying the video configuration layout defined by the video mode of the three video data streams on four video displays simultaneously.

It is another object of the subject matter to disclose a medical imaging system comprising a monitor, and a medical imaging endoscope comprising three cameras adapted to capture a front video stream and two side video streams, and a handle, wherein the medical imaging system comprises a control means configured to receive commands from a user of the medical imaging system and send control commands to a processing unit, wherein the processing unit is operatively connected with the medical imaging endoscope and the monitor, wherein the processing unit comprises a circuitry board configured to perform a video interpolation on each video stream of the front video stream and the two side video streams based on a video mode in order to display a video configuration on the monitor, wherein the video configuration comprises three video displays concurrently to provide a panoramic view based on an overlap between field of views of the three cameras, wherein the processing unit is utilized to receive the front video stream and the two side video streams into a single, panoramic video frame, based on an overlap between field of views of the three cameras; wherein the video configuration comprises and display on a first area of the monitor a larger centric video display and display on a second area of the monitor one of the three video streams, according to the video mode.

In some cases, the first area of the monitor is an upper area of the monitor and the second area of the monitor is a lower area of the monitor. In some cases, the first area of the monitor is a lower part of the monitor and the second area of the monitor is an upper part of the monitor.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 demonstrates a medical imaging endoscope comprising a distal tip with at least two cameras, according to exemplary embodiments of the disclosed subject matter;

FIG. 2 shows a medical imaging system, according to exemplary embodiments of the disclosed subject matter;

FIG. 3 shows a schematic view of a medical imaging endoscope with a changing diameter comprises one front camera and two side cameras, according to exemplary embodiments of the disclosed subject matter;

FIG. 4 is a block diagram depicting an embodiment of an overall video processing architecture of a medical imaging system connected to a rigid shaft, according to FIG. 2;

FIG. 5A discloses a medical imaging system configured to administrate a layout of a first video configuration in accordance with one optional layout defined by a first video mode, in accordance with exemplary embodiment of the disclosed subject matter;

FIG. 5B discloses a medical imaging system configured to administrate a layout of a second video configuration in accordance with another optional layout defined by a second video mode, according to FIG. 5A;

FIG. 5C discloses a medical imaging system configured to administrate a layout of a third video configuration in accordance with another optional layout defined by a third video mode, in accordance with exemplary embodiment of the disclosed subject matter;

FIG. 5D discloses a medical imaging system configured to administrate a layout of a fourth video configuration in accordance with another optional layout defined by a fourth video mode, in accordance with exemplary embodiments of the disclosed subject matter;

FIG. 5E discloses a medical imaging system configured to administrate a layout of a fifth video configuration in accordance with another optional layout defined by a fifth video mode, in accordance with exemplary embodiments of the disclosed subject matter;

FIG. 6A discloses a medical imaging system configured to administrate a layout of a video configuration comprising more than three displays, in accordance with exemplary embodiments of the disclosed subject matter;

FIG. 6B discloses a medical imaging system having a non-flat monitor and configured to administrate a layout of a video configuration comprising more than three displays, according to FIG. 6A, and

FIG. 7 illustrates a flow chart illustrating the steps of displaying a selected video configuration of a selected video mode on a monitor of a medical imaging system, in accordance with embodiments of FIGS. 5-6.

### DETAILED DESCRIPTION OF THE INVENTION

The subject matter in the present invention discloses a medical imaging endoscope comprising a multi camera rigid endoscope designs to aid medical procedures such as inspection or surgery procedures within for example and not limited to the abdomen or pelvis through small incisions made into the body. Such imaging endoscope may comprise two or more cameras designed to aid medical procedures such as inspection or surgery.

The term “optical gear module” or “optical gear” is used herein to depict a set of components that allow the medical imaging endoscope to capture light and transform that light into at least two images. In some embodiments, lenses are employed to capture light, image capturing devices, such as sensors, are employed to transform that light into at least one image and illumination modules are employed to provide light. In some embodiments, a camera comprises a plurality of optics such as lens assembly and sensor and is configured to receive reflected light from target objects. In some embodiments, an optical gear located in a distal tip of the medical imaging endoscope can comprise a sensor and lenses (e.g., camera) and light sources required for the sensor functioning.

Image capturing devices may be Charged Coupled Devices (CCD's) or Complementary Metal Oxide Semiconductor (CMOS) image sensors, or other suitable devices comprising a light sensitive surface usable for capturing an image. In some embodiments, a sensor such as a Charge Coupled Device (CCD) or a Complementary Metal Oxide Semiconductor (CMOS) image sensor, for detecting the reflected light received by an optical element, is employed.

It should also be noted that a plurality of terms, as follows, appearing in this specification are used interchangeably to apply or refer to similar components and should in no way be construed as limiting: A “medical imaging device” may also be referred to as a “medical imaging endoscope”. A “camera” may also be referred to as an image capturing device/component, comprises a lens assembly and sensor. An “illuminator” may also be referred to as an “illumination source”. Illuminators may optionally be discrete illuminators and may include a light-emitting diode (LED), which may be a white light LED, an infrared light LED, a near infrared light LED, an ultraviolet light LED or any other LED and or combination of the same. A “niche” may also be referred to as a “shallow trough”. A “video data stream” may also be referred to as a “video stream”.

It is noted that the term “endoscope” as mentioned to herein may refer particularly to a rigid scope, semi-rigid scope or flexible scope such as laparoscope, according to some embodiments of the disclosed subject matter, but is not limited only to laparoscope, and may include other applications such as industrial applications. The term “endoscope” may also refer to any instrument used to examine the interior of a hollow organ or cavity of the body.

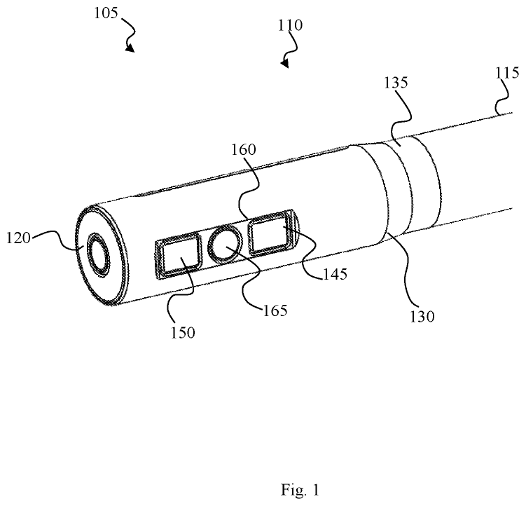

FIG. 1 demonstrates a medical imaging endoscope comprising a distal tip having at least two cameras, according to exemplary embodiments of the disclosed subject matter. FIG. 1 shows a medical imaging endoscope 105 comprising a distal tip component 110 designed to be directly connected to a rigid shaft 115. The distal tip 110 may comprise an inclined surface 130 allowing to connect the distal tip 110 to the rigid shaft 115 provided in a narrower diameter than the diameter of the distal tip 110. The medical imaging endoscope 105 also comprises a seamline 135 which outlines the connection line between the rigid shaft 115 and the inclined surface 130 of distal tip component 110. In some cases, the rigid shaft 115 and the distal tip component 110 may be connected by an adhesive material that seals the connection at the seamline 135. In some other cases, the rigid shaft 115 and the distal tip component 110 may be connected by welding or soldering. In possible embodiments of the disclosed subject matter, the rigid shaft 115 and the distal tip component 110 may be connected by a screwing mechanism which fastens the rigid shaft 115 and the distal tip component 110 together.

In some cases, the distal tip component 110 may function as a multi camera section member designed to house at least two cameras. The distal tip 110 may comprise a front camera located at a front end of distal tip 110, denoted as a planar surface 120. Additional cameras may be located at the lateral round surface of the distal tip component 110. In some embodiments, the front end of distal tip 110 has a tilted angle adapted to provide a tilted front surface. The cameras can be configured to aid medical and surgery procedures within for example and not limited to, the abdomen or pelvis through small incisions made into the body. In some cases, such medical imaging device 105 can be utilized in laparoscopy wherein the medical imaging device can be inserted through a small incision in the body in order to perform medical procedures at the internal organs.

The distal tip component 110 may also comprise a niche or a shallow trough 160 designed to house a first side camera optical window 165 and provide the opening required for the field of view of a first side camera (not shown). In some cases, the first side camera optical window 165 may comprise a transparent layer, such as glass or plastic, to isolate the first side camera (not shown) from liquids, gases and patient's debris and tissue. In some other cases, first side camera (not shown) may be covered by an optical window or more than one optical window. The niche 160 also enables emission of light from first side illuminator/s windows 150, and 145. In some cases, the light may be emitted by dedicated section illuminators such as light-emitting diodes, also known as LED. The dedicated section illuminators may be housed in windows 150, and/or 145. In some cases, the first side illuminator/s windows 145 and 150 may comprise a transparent layer, such as glass or plastic, to isolate the side illuminator/s from liquids, gases and patient's debris and tissue. In some other cases, second side illuminator/s windows 145 and 150 may comprise an optical window or more than one optical window. In yet some other embodiments, niche 160 may be covered by a transparent layer, such as glass or plastic or an optical window or more than one optical window for isolating niche 160 from liquids, gases and patient's debris and tissue.

The distal tip component 110 may further comprise a second side niche or a shallow trough (not shown) placed 180 degrees or less to the niche 160 along distal tip 110 longitudinal axis and wherein the second side niche is designed to house a second side optical gear module (not shown).

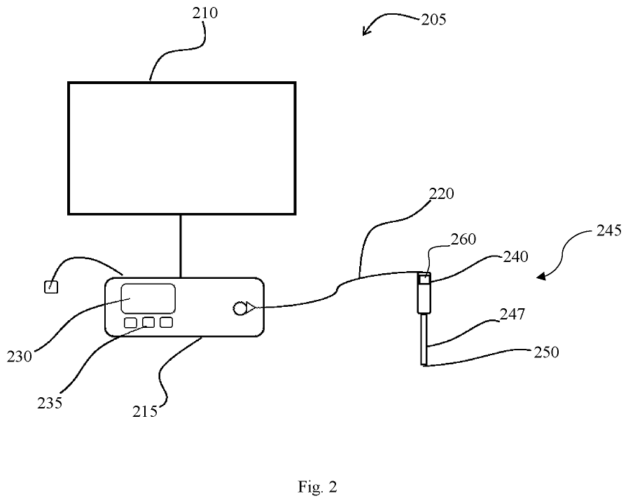

FIG. 2 shows a medical imaging system, according to exemplary embodiments of the disclosed subject matter. Medical imaging system 205 may comprise a display unit 210, and a processing unit such as a main control unit 215 designed to be connected to a medical imaging endoscope 245 by a utility cable 220. The medical imaging endoscope 245, shown in this exemplary embodiment comprises elongated rigid shaft 247 which can be as an endoscope comprising a handle 240. The rigid shaft 247 can be connected to handle 240 in a detachable manner or as one unit. The rigid shaft 247 terminates with a distal tip section 250. The distal tip 250 may comprise a front camera and at least one side camera. The cameras of the distal tip 250 can be configured to transmit the video content captured thereof to the main control unit 215 via at least two video data streams. Such at least two video data streams can be sequences of images processed electronically into an analog or digital format for being displayed on display unit 210 or on more than one display unit with sufficient rapidity as to create the illusion of motion and continuity.

In some cases, the control unit 215 can be configured to receive two or more separated video data streams, process the multiple video data streams (for example as mentioned above, the at least two video data streams) to obtain a video mode to be displayed on the display unit 210 as a video configuration. Processing the multiple video streams may comprise performing an interpolation process on at least one of the multiple video streams to fit a predefined video configuration selected from a plurality of optional video configurations. Such, the video configuration may be a presentation of the at least two video data streams received by the two or more cameras located in the distal tip 250 (not shown) and processed by the main control unit 215 according to a pre-defined video mode. The video mode may character each video data stream in different size, scale, magnitude, frame rate, video display aspect ratio, and the like. Thus, the video configuration can be a combination of separated at least two video data streams received from more than one camera.

The control unit 215 can also be configured to govern the video mode to be displayed on the display unit 210 in more than one video configuration. The video mode defines the layout of the video configuration. For example, the video mode can define a layout of three separated video contents streamed from three different cameras, wherein the three separated video contents are in a layout wherein each video content is displayed next to the other, and wherein the video contents are displayed at same size. In some other video modes, the layout of the video contents can be with different video contents displayed with different sizes, or different scales. In some embodiments of the present invention the video modes may be predefined templates controlled by the control unit 215. For example, the control unit 215 may be configured with a predefined template of a video mode which defines one video content displayed in the center of the display unit 210, in a relatively larger scale than other video displays, and two video displays presented in each side areas of the video content displayed in the center, wherein the two video contents are displayed in smaller scales or sizes relative to the video contents displayed in the center.

Handle 240 may be utilized for guiding rigid shaft 247 within a body cavity. In some cases, the handle 240 may comprise handle board 260 comprising control means such as one or more buttons and/or switches and/or knobs. The handle board 260 can be configured to control commands, as well as zoom, focus, record and the like. In some cases, the handle board 260 may also be utilized to change the layout of the video configuration. For example, when utilized the rigid shaft 247 by controlling thereof with the handle 240 can utilize the handle board 260 to change the layout of the video configuration from a layout comprising three separated video contents to a layout comprising one video content. In some cases, the handle board 260 may be configured to allow choosing diverse video modes and thereby to change the layout of the video configuration.

The distal tip section 250 may comprise at least one optical gear module providing the user with a field of view necessary for examine the region of interest as well as medical instruments and other organs surround the treated area. In some embodiments, the field of view of each of the at least one optical gear module may be of at least 60 degrees, at least 80 degrees, at least 100 degrees, at least 120 degrees. In some embodiments, a depth of field of each of the at least one optical gear module may be of approximately 15 to 150 millimeters, of approximately 5 to 50, of approximately 5 to 150.

The utility cable 220, may connect between handle 240 and the main control unit 215. The utility cable 220 may comprise therein one or more electrical channels. The electrical channel(s) may comprise at least one data cable for receiving video signals from the at least one optical gear module of the distal tip. In an embodiment, the electrical channel(s) may comprise at least one data cable for receiving at least two video data streams from a front and at least one side cameras, as well as at least one power cable for providing electrical power to the cameras and to the illuminators associated with the at least one optical gear module. In another embodiment of the present invention, wireless communication between handle and main control unit can be configured and utilized.

The main control unit 215 comprises controls, such as controls 235, required for displaying the images of internal organs captured by the optical gear module/s of distal tip section 250. In some cases, the main control unit 215 may comprise functionalities such as but not limited to handle board 260, and or controls 235, and or a touch screen, and or a voice controller for changing the layout of the video configuration.

The main control unit 215 may direct power transmission to the endoscope's distal tip 250 components, such as for the camera/s and illuminators. One or more input devices, such as a keyboard, a mouse, a touch screen, and the like may be connected to the main control unit 215 for the purpose of controlling thereof. In the exemplary embodiment shown in FIG. 2, the main control unit 215 comprises a display 230 for displaying operational information concerning the operation and activities of the medical imaging endoscope 245. The operational information may be changed and/or determined by operation buttons, voice control and the like which may also be located on the main control unit 215 and or on handle board 260. The images/videos may be displayed separately on one or more display units, such as display unit 210 by uploading information from the main control unit 215, either side-by-side or interchangeably, namely, the user operate the main control unit 215 may switch between views from the different viewing elements manually. Alternatively, these at least two video data streams may be processed by the main control unit 215 to incorporate these received at least two video data streams into a multiplex and video configuration.

The main control unit 215 may also be designed to receive the digital inputs from the distal tip 250 of medical imaging endoscope 245 and convert said digital input to a video data stream/s displayed on the display unit 210. The display unit 210 may further be operative to display a user interface for allowing an operator to set various features of the endoscopy system. In some cases, the main control unit 215 may also comprise a controller (not shown) programmed to interlock with the medical imaging endoscope 245 and configured to control some operational tasks of the medical imaging endoscope 245. The operational tasks may such as: start/stop recording the field captured by the optical gear module/s, start/stop insufflation, start/stop LEDs activity, report the state of the optical gear module/s to an external system, and the like. Thus, the controller may also comprise a software unit operable on a processor and designed to execute the instructions stored in the computer-readable storage medium. The controller may further comprise a synchronization component adapted to generate synchronization signals to synchronize video data/stream received from cameras placed in the distal tip of the endoscope. For example, in some embodiments, synchronization component synchronizes video data received through the cameras by generating a common clock signal and driving the sensor of each of the cameras with the common clock signal. In some embodiments, synchronization component synchronizes video data received through the cameras by generating an initiating synchronization signal initiating the scan in each sensor at the same instant. In some cases, the controller may also comprise an independent base board module and a Field Programmable Gate Array (FPGA), or in some cases an Application Specific Integrated Circuit (ASICS), configured to implement an algorithm to detect changes in Red Grin Blue (RGB) image colors, or other image colors, obtained by the plurality of optical gear modules. In some embodiments of the present invention, the medical imaging system 205 may be configured to allow a user utilizing the medical imaging endoscope 245 to control the operational tasks from the handle board 260.

In some embodiments of the present invention, the controller may be just a software unit operable on a processor and configured to interlock with the camera rigid endoscope. For example, the control unit 215 may be implemented with a processor and the controller may be operable on the processor of the control unit.

In some cases, the main control unit 215 may also be configured to execute the operational tasks, according to a predefined set of conditional events associated with the rigid shaft 247 positions. For example, the main control unit 215 may start recording a field of view captured by one or more of the optical gears. In some cases, the operational tasks may be controlled by the main control unit 215, according to the change detection results as received from the controller. In some other cases, the operational tasks may be controlled by the main control unit 215, according to a computerized input indicating the position of the rigid shaft 247 of medical imaging endoscope 245. For example, the main control unit 215 may receive a computerized input received from an input device and indicating the position of the medical imaging endoscope 245, wherein the computerized input indicates that the rigid shaft 247 is within a patient body. In such an exemplary case, the main control unit 215 may perform the operational tasks as define above, according to a predefined set of conditional events.

In some embodiments of the present invention, the controller may be located at handle 240. In such embodiments of the specification, the controller may comprise a processor and a computer-readable storage medium for executing the instruction. The controller located at handle 240 may also be configured to communicate with system main control unit 215.

In some cases, the main control unit 215 may further comprise an image processing module (not shown) which can conduct a synchronized transferring of one or more video signals from the one or more cameras of the medical imaging endoscope 245 to the display unit 210. The image processing module may also be programmed to control a record of the at least one of optical gear module's video frame/image and or a video signal.

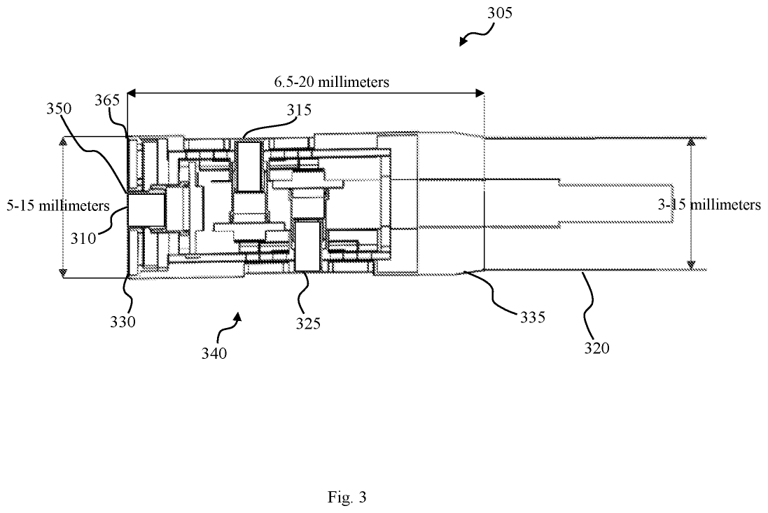

FIG. 3 shows a schematic view of a medical imaging endoscope with a changing diameter comprises one front camera and two side cameras, according to exemplary embodiments of the disclosed subject matter. FIG. 3 shows a medical imaging endoscope 305 comprising a distal tip 340 designed to house three cameras. The distal tip 340 may house a front camera 310 positioned essentially at the center of the front planar surface 350. In some embodiments, front camera 310 may not be positioned at the center of front planar surface 350, and such may be situated with a bias to one of the sides of the front planar surface 350. In some embodiments, the field of view of front camera 310 may be of at least 60 degrees, at least 80 degrees, at least 100 degrees, at least 120 degrees. In some embodiments, a depth of field of front camera 310 may be of approximately 15 to 150 millimeters, of approximately 5 to 50, of approximately 5 to 150.

The distal tip 340 further comprises a second side camera 325 positioned/located at the lateral surface of the distal tip 340. The distal tip 340 further comprises a first side camera 315. The first side camera 315 may be positioned/located at the opposite lateral surface of the second side camera 325 such that the two cameras second side camera 325 and first side camera 315 may be pointing at directions essentially opposing to one another. In some other cases, the angle between the second side camera 325 and first side camera 315 may be any workable angle in the range of 10-180 degrees on the circumference of the distal tip 340. In some embodiments, the field of view of second side camera 325 may be of at least 60 degrees, at least 80 degrees, at least 100 degrees, at least 120 degrees. In some embodiments, a depth of field of second side camera 325 camera 310 may be of approximately 15 to 150 millimeters, of approximately 5 to 50 millimeters, of approximately 5 to 150 millimeters. In some embodiments, the field of view of first side camera 315 may be of at least 60 degrees, at least 80 degrees, at least 100 degrees, at least 120 degrees. In some embodiments, a depth of field of first side camera 315 camera 310 may be of approximately 15 to 150 millimeters, of approximately 5 to 50 millimeters, of approximately 5 to 150 millimeters.

The distal tip 340 can be provided in a diameter of approximately 2.5 to 15 millimeters. The length of the distal tip 340 may be approximately 6.5 to 20 millimeters. In some cases, the distal tip 340 can be connected to a rigid shaft 320, which can be have a diameter of approximately 2.5 to 15 millimeters. In possible embodiments of the disclosed subject matter, the diameter of the rigid shaft 320 may be narrower than the diameter of the distal tip 340. Thus, the distal tip 340 may comprise an inclined surface 335 allowing to connect the distal tip 340 to the rigid shaft 320 provided in a narrower diameter.

In possible embodiments of the disclosed subject matter, the distance between the center of the second side camera 325 and a front surface second edge point 330 may be at the same distance between the center of the first side camera 315 and a front surface first edge point 365. In possible embodiments of the disclosed subject matter, the distance between the center of the second side camera 325 and the second edge point 330 may be shorter than the distance between the center of the first side camera 315 and the first edge point 365. In possible embodiments of the disclosed subject matter, the distance between the center of the second side camera 325 and the second edge point 330 may be longer than the distance between the center of the first side camera 315 and the first edge point 365.

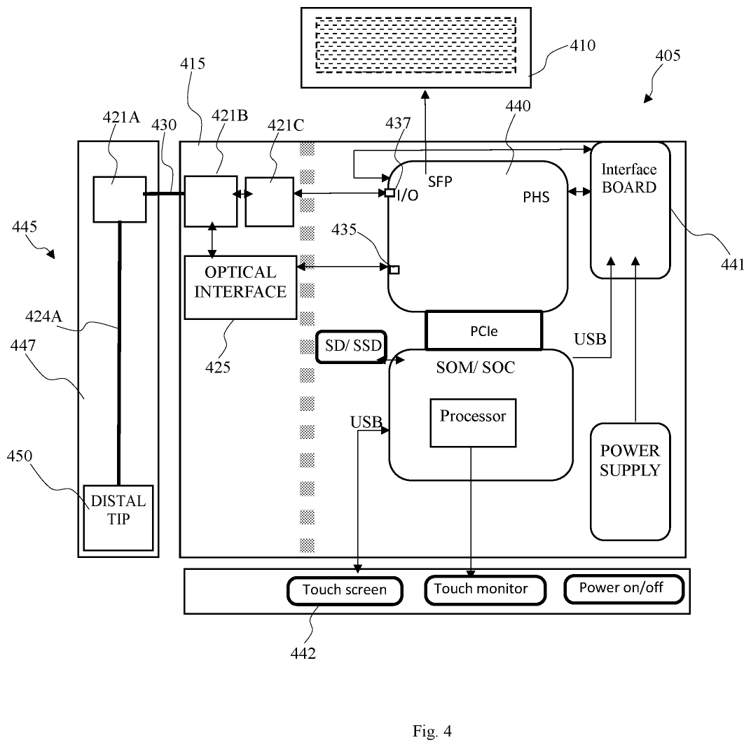

FIG. 4 shows a schematic view of a medical imaging system wherein a main control unit operatively connects with a medical imaging endoscope and a monitor, according to FIG. 2. FIG. 4 shows a medical imaging system 405 comprises a processing unit such as a main control unit 415 operatively connects with a medical imaging endoscope 445 and a monitor 410. The medical imaging endoscope 445, shown in this exemplary embodiment comprises elongated rigid shaft 447 terminates with a distal tip section 450. The distal tip 450 comprises at least two optical gears of which at least one front optical gear and at least two side optical gears are placed within distal tip 450. Each optical gear has a camera and illuminators that operates by commands transmit by a handle board 421A located also within endoscope 445. The camera or the cameras of the distal tip 450 can be configured to transmit the video view captured thereof to the main control unit 415. In some cases, the control unit 415 can be configured to perform video interpolation on each video data stream follow by transmitting pre define video modes in order to display aligned video configurations to the monitor 410.

The handle board 421A comprises control means such as one or more buttons and/or switches (not shown). The control means, such as buttons and/or switches of the handle board 421A, can be configured to send control commands such as zoom, focus, record and the like to the main control unit 415 and such to align the display on display unit 410 accordingly and or start/stop recording the procedure. In some cases, the buttons and/or switches of the handle board 421A may also be configured with the required control commands for changing the layout of the video configuration. For example, when utilized the rigid shaft 447 can utilize the handle board 421A to change the layout of the video configuration from a layout comprising three video streams to a layout comprising one stitched video stream. In some embodiments, main control unit 415 may utilize the received video streams into a single, panoramic video frame, based on an overlap between field of views of the cameras.

The medical imaging endoscope 445 can also be configured to transmit the digital video content captured by the camera/s located at the distal tip 450 to the main control unit 415. The main control unit 415 can be configured to receive the video content transmitted from the cameras. In some cases, the video content may be received by a handle interface 421B. The handle interface 421B can be configured to transmit the video streaming from the cameras of the distal tip 450, which generated the video streaming. Such a video streaming can be transmitted by a utility cable 424A to the handle board 421A. The utility cable 424A may comprise therein one or more electrical channels. The electrical channel(s) may comprise at least one data cable for conveying video streaming received from the cameras of the distal tip 450 to the handle board 421A. The electrical channel(s) of the utility cable 424A may also comprise at least one power cable for providing electrical power required for the functioning of the optical gear modules of the distal tip 450 and such supply power and control the operation of the illuminator/s of the optical gear modules.

The handle board 421A can be configured to receive at least two parallel separated video data streams transmit by the utility cable 424A and send a serial video data stream via a utility cable 430 and or wireless transmission to the handle interface 421B. In some cases, the handle board 421A can be configured to process the received at least two video data streams. For example, the handle board 421A can be configured to perform the serialization of the at least two video data streams into one or more serial lines. In some cases, the handle board 421A may comprise a processing unit encoded with the computerized instructions required for the process of the received at least two video data streams. For example, the handle board 421A can receive three video contents provided by three separated video data streams generated by the cameras. The handle board 421A can be a Field Programmable Gate Array (FPGA), or in some cases an Application Specific Integrated Circuit (ASICS), configured to serialize the at least two parallel video data streams into serial lines and then transmit the one, two, three or more video data streams further by utilizing the utility cable 430. In some embodiments, the at least two video data streams are wireless transmission from handle board 421A to the handle interface 421B. The handle board 421A in return receives one, two, three or more parallel video signals generated by the one, two, three or more image sensors and also other remote commands from the control unit 415. Other remote commands may include zoom, focus, record and the like.

In some embodiments of the disclosed subject matter, the handle interface 421B can be configured to split the video streaming and the control commands received from the handle board 421A and route control commands to a circuitry board 440. For example, the handle board 421A can be connected to an I/O port 437 for transmitting the control commands to the circuitry board 440. The circuitry board 440 can be a Field Programmable Gate Array (FPGA), or in some cases an Application Specific Integrated Circuit (ASICS). The handle interface 421B can also be configured to receive the one or more serial lines and transmit a single serial video stream that compose the data of the one, two, three or more serial lines to an optical interface 425. The optical interface 425 may also be designed to handle electrical signals representing the video content received via one, two, three or more video streams sent from the handle board 421A.

The optical interface 425 may also be designed to convert the electrical signals representing the content of the one, two, three or more video streams and convert thereof to video computer-readable content. The computer-readable content can be such as analog video, such as digital video (DVI). In some embodiments of the disclosed subject matter, the optical interface 425 can be configured to convert the signals representing the video content to the video computer-readable content can be other digital data structures used to connect a video source, wherein the video content is represented by the digital data structure. The optical interface 425 can be configured to transmit the one, two, three or more video streams of the circuitry board 440. For example, the optical interface 425 can transmit the video contents to the optical port 435 via an optical port 435.

In some embodiments of the disclosed subject matter, the handle interface 421B can also be configured to receive control commands sent from the handle board 421A and transmit the received control commands to the non-optical interface 421C. In some embodiments of the disclosed subject matter, the handle interface 421B, the optical interface 425, and the non-optical interface 421C may be located on one integrated circuit. Such an integrated circuit may comprise a processing unit instructed with the computerized insurrections required for the operational tasks of the handle interface 421B, the optical interface 425, and the non-optical interface 421C. For example, the integrated circuit comprising the handle interface 421B, the optical interface 425, and the non-optical interface 421C can be a Field Programmable Gate Array (FPGA), or in some cases an Application Specific Integrated Circuit (ASICS), configured to process the one, two, three or more video data streams to one data stream and the control commands as aforementioned.

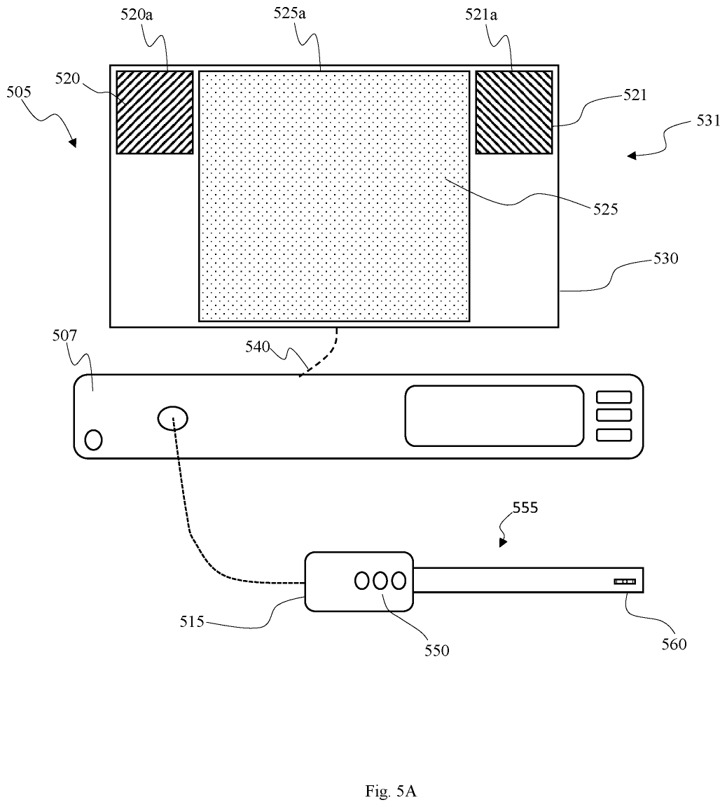

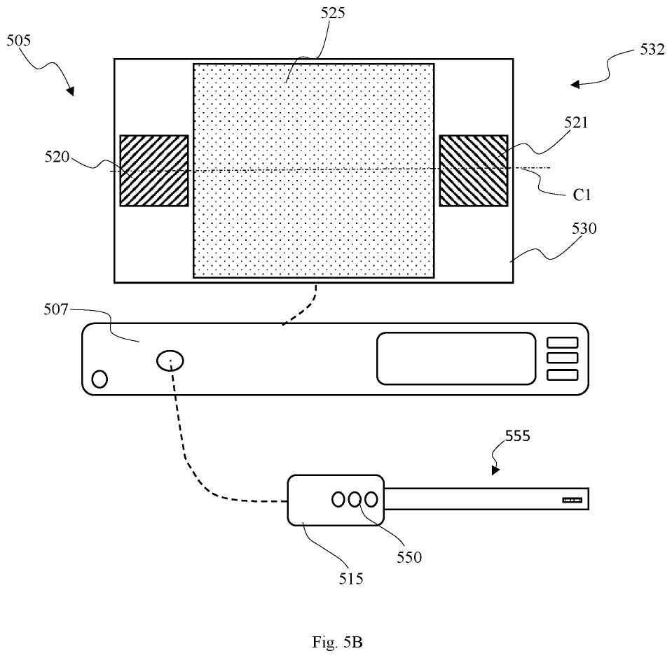

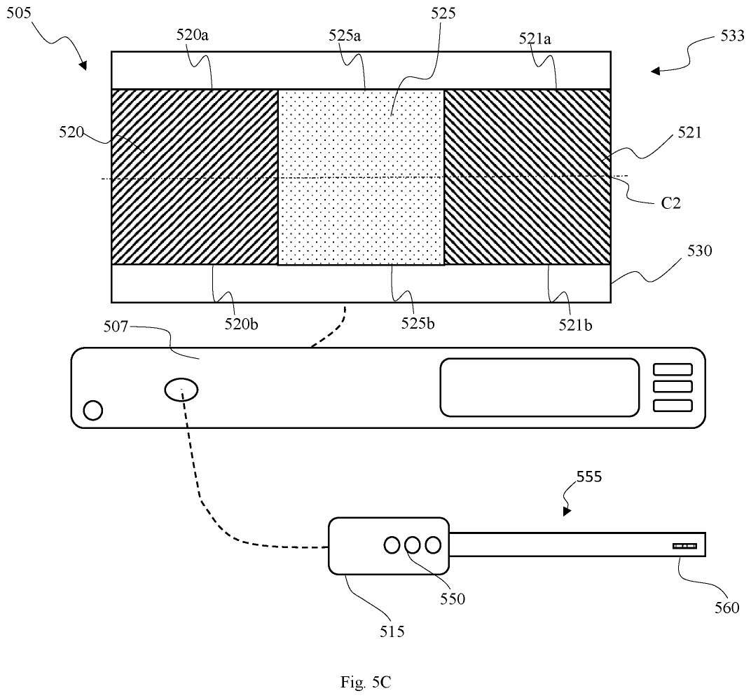

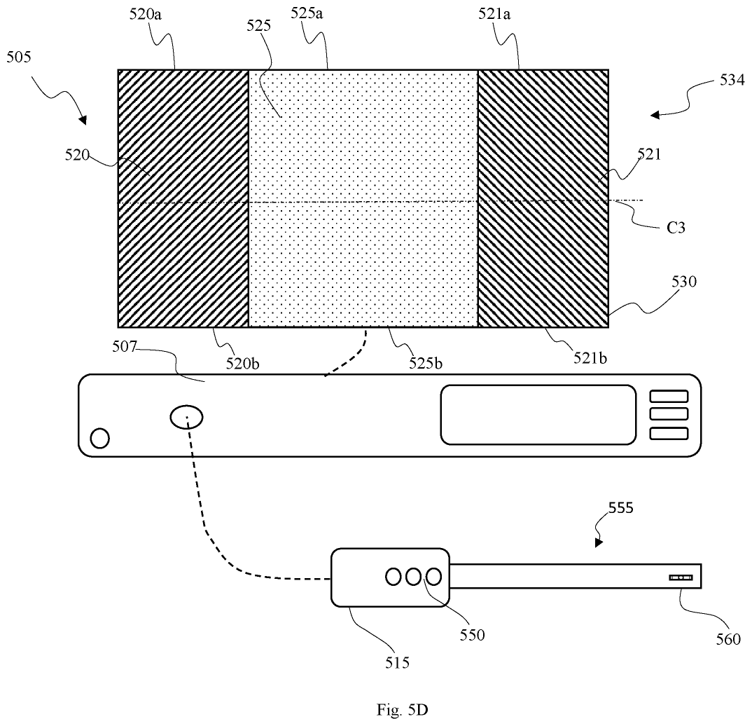

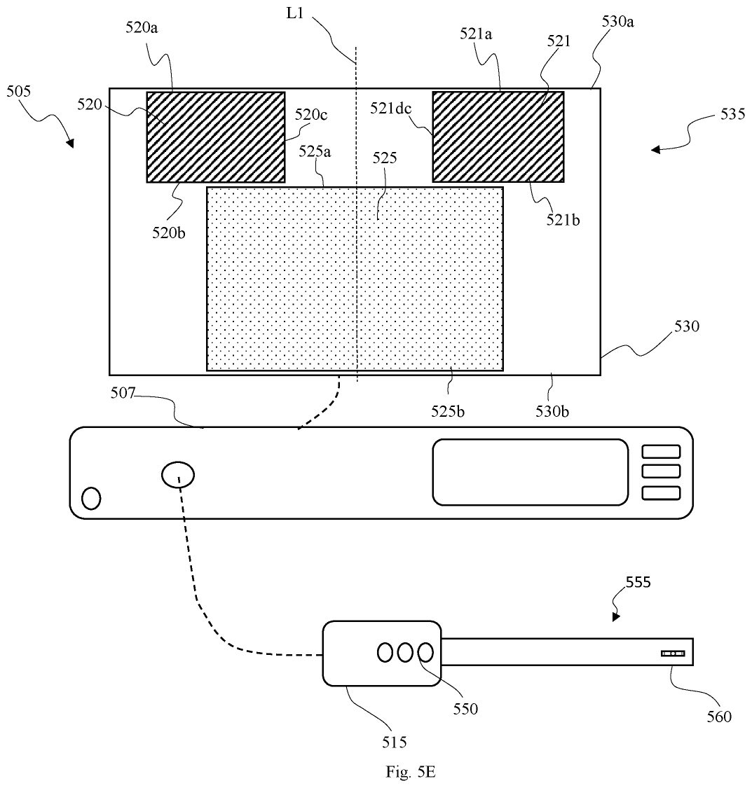

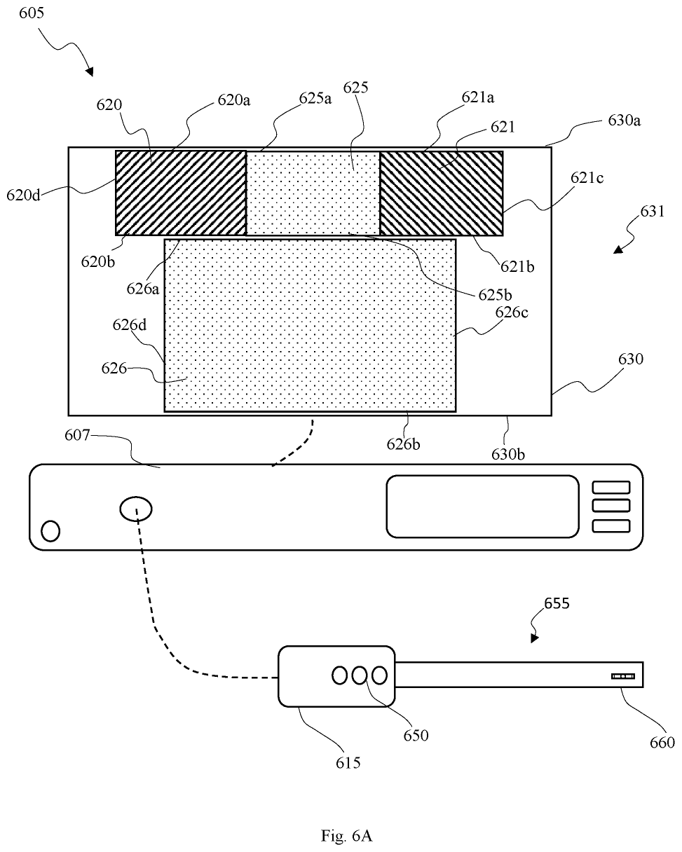

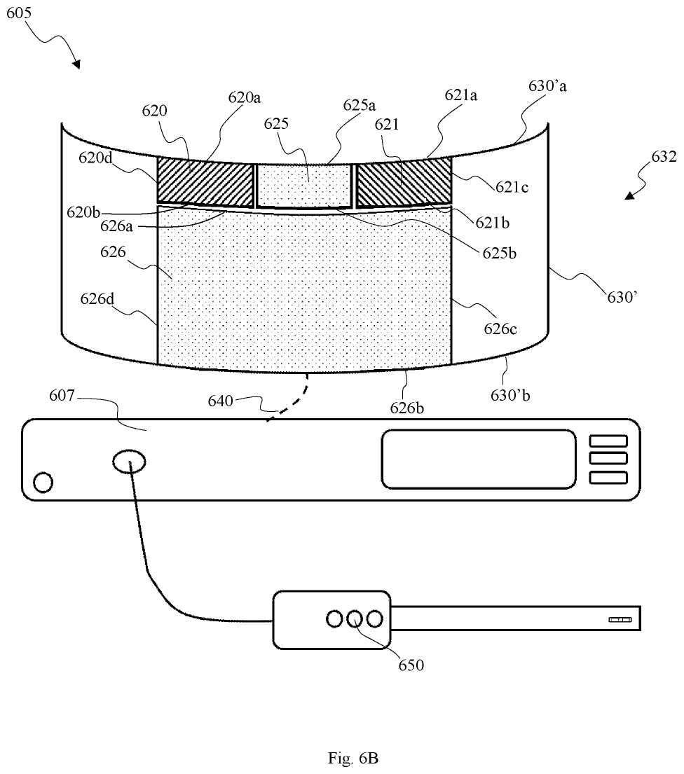

In some embodiments of the present invention, the circuitry board 440 can be configured to receive the control commands via I/O port 437 and the video computer-readable data received via the optical port 435. The circuitry board 440 may configured to perform a video interpolation on each video stream followed by a pre-set of video modes in order to display aligned video configuration on the monitor 410. The video interpolation may be performed independently on each of the video streams. In some cases, the video configuration mode may be prepared in accordance with the control commands received from the I/O port 437. For example, the command control may comprise a command to obtain the video configuration according to a specific/dedicated video mode. Exemplary display formats are introduced in FIGS. 5 to 6. Thus, the circuitry board 440 can be configured to receive the video streams and administrate the layout of the video configuration according to the video mode chosen by user operating medical imaging system 405.

In some embodiments, circuitry board 440 may include a synchronization component in communication with handle board 421A. The synchronization component is adapted to generate synchronization signals to synchronize video data/stream received from cameras placed in distal tip 450 of endoscope 445. For example, in some embodiments, synchronization component synchronizes video data received through the cameras by generating a common clock signal and driving the sensor of each of the cameras with the common clock signal. In some embodiments, synchronization component synchronizes video data received through the cameras by generating an initiating synchronization signal initiating the scan in each sensor at the same instant.

In some embodiments of the disclosed subject matter, the circuitry board 440 can be configured to utilize video interpolation for structuring the video configuration in accordance with the chosen video mode. In such cases, the circuitry board 440 can utilize video interpolation comprising parameters which define the characterizations of the displays. Such parameters can be for example and not limited to, the number of video frames to output, the frame rate (Hz value, fraction or abbreviation), timestamps assuming constant frame rate, video display aspect ratio specified by aspects, such as and not limited to 4:3, 16:9, 16:10, 32:9, pixel format defining the memory layout of each pixel in a bitmap, overlap lines and magnitudes, and more. Thus, the circuitry board 440 can be configured to receive data of one, two, three or more video streams, follow by performing a video interpolation according to a pre-set of video modes for obtaining video configurations. The video interpolation may be performed independently on each of the video streams. In some embodiments, circuitry board 440 may utilize the received video streams into a single, panoramic video frame, based on an overlap between field of views of the cameras.

In one embodiment, circuitry board 440 has a direct connection to monitor 410 through HDMI and or SDI and or DVI lines, using SFT components. In another embodiment, an interface board, such as interface board 441 is adapted to receive data from a PHS component placed within circuit board 440 and connect to monitor 410 through HDMI and or SDI and or DVI lines. In yet another embodiment, monitor 410 wirelessly communicate with main control unit 415.

FIG. 5A discloses a medical imaging system configured to administrate the layout of a first video configuration in accordance with one optional layout defined by a first video mode, in accordance with exemplary embodiments of the disclosed subject matter. FIG. 5A shows a medical imaging system 505 comprises a processing unit such as a main control unit 507 operatively connected with a medical imaging endoscope 555 and with a monitor 530 via communication cable 540 or via wireless communication. The operation of the main control unit 507 is disclosed at FIG. 4. The monitor 530 can be a display device such as electronic visual display. The medical imaging system 505 can be configured to transmit one, or more video mode of video data stream and display those simultaneously, wherein video mode may character each video data stream in different size, scale, magnitude, frame rate, video display aspect ratio, and the like. The monitor 530 presents a first video configuration 531 of a first video mode displays three video streams respectively associate with a front camera and two side cameras located at a distal tip 560 of a medical imaging endoscope 555. First video configuration 531 comprises a right video display 521 located at the upper right side/area of monitor 530, a centric video display 525 located at a center area of monitor 530, and a left video display 520 located at the upper left side/area of monitor 530. The first video configuration 531 displays the right video display 521, the centric video display 525, and the left video display 520 simultaneously at the monitor 530, such that the respectively upper edges 521a, 525a, 520a are at a substantially same level.

In one embodiment, the two side video displays 521 and 520 corresponding to the two side cameras are processed for display such that the right video display 521 corresponds to right side camera is displayed right aligned on the upper right side/area of monitor 530, and left video display 520 corresponds to left side camera is displayed left aligned on the upper left side/area of monitor 530. In one embodiment, the centric video display 525 corresponding to front camera is processed for display such that centric video display 525 is displayed centric of the monitor 530.

The first video configuration 531 of the first video mode may be controlled by control means such as button/knob/switch set 550 placed within a handle 515 of medical imaging endoscope 555. The button/knob/switch set 550 can be configured with the required buttons and computerized equipment required to send control commands to the medical imaging system 505. Such control commands can comprise instructions to change the video mode of the video configuration presented on the monitor 530. In some cases, the button/knob/switch set 550 is configured with the required commands to change the video mode of the video configuration presented on the monitor 530, by utilizing alternative predefined video modes. In some cases, such video modes can comprise data and/or instructions about the size, scale, magnitude, frame rate, video display aspect ratio and more of the video configurations and the locations thereof. In some embodiments, first video configuration 531 may be controlled by control means such as buttons, knobs, touch screen, voice control and the like placed within main control unit 507.

In some cases, the video interpolation process is performed at main control unit 507 for at least one of the two or more video streams. The video interpolation process performed on multiple video streams may be either identical or different. For example, video configurations to be displayed on the display device (monitor) dictate that at least a portion of the parameters defining the characteristics of each video stream be different for each of the video streams. Such parameters may include size, scale, magnitude, video display aspect ratio and the like can. The characteristics of the video interpolation process define the manner of displaying each video stream on the monitor. For example, in case the main control unit 507 receives 3 video streams, the interpolation process may be identical for streams #1 and #3 and have a different aspect ratio for stream #2, based on characteristics stored in a memory module of the device. In such exemplary case, the magnitude of the side video displays 521 and 520 can be different from the magnitude of the video display 525 displayed in the center. In some other exemplary cases, the scale, the size, the frame rate, and the like, can be different among the video displays 521, 525 and 520, wherein the displays 521, 525 and 520 are played simultaneously.

In some cases, the sizes of the video displays can be different one from each other. For example, the height and the width of the video displays 521 and 520 can be one-third of the height and the width of video display 525. In another example, the height and the width of the video displays 521 and 520 can be half of the height and the width of video display 525. In yet some other examples, the height and the width of the video displays 521 and 520 can be a quarter of the height and the width of video display 525. Persons of ordinary skill in the art should appreciate that the height and the width of the right and left video displays 521 and 520 relatively to the height and the width of centric video display 525 may include other values such as but not limited to 1:1.5, 1:2.5, 1:2.2, 1:3.5, 1:3.7 and more.

In some cases, the video mode may also comprise parameters and/or instructions regard the characterizations of the displays such as magnitude, the scale, the size, the ratio, and the like. For example, a user holding the handle 515 can use the button/switch set 550 to change the characterizations of the displays and or the user may use the control means placed within main control unit 507 to change the characterizations of the displays. The characterizations of the displays can be such as, the number of video frames to output, the frame rate (Hz value, fraction or abbreviation), timestamps assuming constant frame rate, video display aspect ratio specified by aspects, such as and not limited to 4:3, 16:9, 16:10, 32:9, pixel format defining the memory layout of each pixel in a bitmap, overlap lines and magnitudes, and the like.

In some cases, the user holding the handle 515 can use the button/switch set 550 and or the control means to change the characterizations of the displays without changing the locations of the displays, or the layout of the unified displayed presentation displayed on the monitor 530. In some cases, the user which may utilize the medical imaging system 505 may use the button/switch set 550 and or and or the control means to record and save the recorded material transmitted by the medical imaging system 505.

In some embodiments of the disclosed subject matter the user operating the medical imaging system 505 may change the video configuration by using predefined video mode saved on a circuit board, such as circuit board 440 of FIG. 4. In some other cases, the user operating the medical imaging system 505 may be able to change one aspect of a displayed video configuration. Thus, the user operating the medical imaging system 505 may be able to change the zoom of the magnitude exclusively in one display, in an ad-hoc fashion. For example, the user operating the medical imaging system 505 may utilize the button/switch set 550 and or the control means to unexpectedly change the zoom or different magnitude of display 521 without changing other aspects of the characterizations of the displays. For example, in some cases, the zoom change may be only on display 525 or display 520.

In some embodiments of the disclosed subject matter, the button/switch set 550 and or the control means may be configured to allow user operating the medical imaging system 505 to change the location of each display on monitor 530. For example, in case display 521 displays video content captured and streamed by a first side camera (not shown), and the display 520 displays video content captured and streamed by a second side camera (not shown), the user utilizing the button set 550 may be able to change the displays locations of the displays 521 and 520 on monitor 530. Such a change of the display locations may cause to display 520 to be seen in the right side/area of the first video configuration 531, right to the display 525 and to display 520 to be seen at the left side/area of the first video configuration 531 left to display 525. In some cases, the button/knob set 550 and or the control means may be configured also to allow user operating the medical imaging system 505 to change the parameters defining the characterizations of the displays. For example, in case displays video content captured and streamed by a front camera (not shown), the button/switch set 550 and or the control means can be configured to allow changing the location of display 525 and the parameters defining the characterizations of the display such that display 525 can be seen at left side/area of monitor 530 instead of the display 520 and the display 520 to be seen at the center area of monitor 530 instead of display 525. In such cases, the display 525 located at the left upper side/area of monitor 530 may be characterized by a smaller size and the display 520 located at the center of monitor 530 may be characterized by a larger size. In some cases, the configuration of the button/switch set 550 may allow other parameters defining the characterizations of the displays to be changeable by user.

FIG. 5B discloses a medical imaging system configured to administrate the layout of a second video configuration in accordance with an optional layout defined by a second video mode, according to FIG. 5A. FIG. 5B shows a medical imaging system 505 comprises a processing unit such as a main control unit 507 operatively connected with a medical imaging endoscope 555 and with a monitor 530 via communication cable 540 or via wireless communication. The operation of the main control unit 507 is disclosed at FIG. 4. The communication cable 540 can be configured and designed to transmit video displays according to diverse video signaling formats, as aforementioned. The monitor 530 presents a second video configuration 532 of a second video mode that displays three video streams respectively associated with a front camera and two side cameras located at a distal tip 560 of a medical imaging endoscope 555. Second video configuration 532 comprises a right video display 521 located at the mid right side/area of monitor 530, a centric video display 525 located at the center of monitor 530, and a left video display 520 located at the mid left side/area of monitor 530. In such cases, second video configuration 532 displays the right video display 521, the centric video display 525, and the left video display 520 simultaneously at the monitor 630, such that the centers of the three video displays 525, 525 and 520 are maintained at a substantially same level C1.

In some cases, control means such as a button/knob/switch set 550 can be configured with the required buttons and computerized equipment required to send control commands to the medical imaging system 505. Such control commands can comprise instructions to change the layout of the second video configuration 532 presented on the monitor 530. In some cases, the button/knob/switch set 550 is configured with the required commands to change the layout of the second video configuration 532 presented on the monitor 530, by utilizing alternative predefined video modes. In some cases, such predefined video modes can comprise data and/or instructions about the size of the video displays and the locations thereof. In some cases, the sizes of the displays can be different one from each other. For example, the height and the width of the video display 521 and 520 can be one-third of the height and the width of display 525. Persons of ordinary skill in the art should appreciate that the height and the width of the right and left video displays 521 and 520 relatively to the height and the width of centric video display 525 may include other values such as but not limited to 1:1.5, 1:2.5, 1:2.2, 1:3.5, 1:3.7 and more.

In some cases, the video interpolation performed at main control unit 507 for the two or more video streams may be either identical or different. For example, parameters defining the video characteristics of each display such as the size, scale, magnitude, video display aspect ratio and the like can be different or identical in different displays, and thereby the size, scale, magnitude, video display aspect ratio and the like of the displays may be different one from another. In such exemplary case, the magnitude of the side video displays 521 and 520 can be different from the magnitude of the video display 525 displayed in the center. In some other exemplary cases, the scale, the size, the frame rate, and the like, can be different among the video displays 521, 525 and 520, when the displays 521, 525 and 520 are played simultaneously.

FIG. 5C discloses a medical imaging system configured to administrate the layout of a video configuration in accordance with another optional layout defined by a third video mode, according to FIG. 5A. FIG. 5C shows a medical imaging system 505 comprises a processing unit such as a main control unit 507 operatively connected with a medical imaging endoscope 555 and with a monitor 530 via communication cable or via wireless communication. The operation of the main control unit 507 is disclosed at FIG. 4. The communication cable 740 can be configured and designed to transmit video displays according to diverse video signaling formats, as aforementioned. The monitor 530 presents a third video configuration 533 of a third video mode displays three video streams respectively associate with a front camera and two side cameras located at a distal tip 560 of a medical imaging endoscope 555. Third video configuration 533 comprises a right video display 521 located at the right side/area of the monitor 530, a centric video display 525 located at the center of monitor 530, and a left video display 520 located at the left side/area of monitor 530.

In third video configuration 533, the centric video display 525 displays the video stream corresponding to the front camera, and the right and left displays 521, 520 display video streams corresponding to the two side cameras of distal tip 560 of medical imaging endoscope 555. In such cases, the third video configuration 533 displays the right video display 521, the centric video display 525, and the left video display 520 simultaneously on monitor 530, such that the centers of the three video displays 521, 525 and 520 are maintained at a substantially same level C2, and also respectively upper edges 521a, 525a, 520a and respectively lower edges 521b, 525b, 520b are at a substantially same level relatively to monitor 530. In some embodiments, respectively upper edges 521a, 525a, 520a and respectively lower edges 521b, 525b, 520b are at a substantially different level relatively to monitor 530, such upper edges 521a, 525a, 520a may be at a first distance relatively to monitor upper edge 530a, while lower edges 521b, 525b, 520b may be at a second distance relatively to monitor lower edge 530b. In such embodiments, first distance may be bigger or smaller than second distance.

In accordance with an embodiment, the three video displays 521, 525 and 520 together provide a panoramic view based on an overlap between field of views of the three cameras, the front camera and the two side cameras. Main control unit 507 may utilize the received three video streams into a single, panoramic video frame, based on an overlap between field of views of the three cameras.

The third video configuration 533 of the third video mode may be controlled by control means such as a button/switch set 550 placed within a handle 515 of medical imaging endoscope 555. In some cases, button/switch set 550 can be configured with the required buttons and computerized equipment required to send control commands to the medical imaging system 505. Such control commands can comprise instructions to change the layout of the video mode of the video configuration presented on the monitor 530. In some cases, the button/knob set 550 is configured with the required commands to change the layout of the video mode of the video configuration presented on the monitor 530, by utilizing alternative predefined video modes. In some cases, such predefined video modes can comprise data and/or instructions about the size, scale, magnitude, frame rate, video display aspect ratio and more of the video configurations and the locations thereof. In some embodiments, third video configuration 533 may be controlled by control means such as buttons, knobs, touch screen, voice control and the like placed within main control unit 507. In one embodiment, when a user selects the third video mode, the user will be provided with the third video configuration 533 such that the sizes of the displays 521, 525, 520 can be equal. For example, the height and the width of the video display 521, 525, 520 can be equal.

In some cases, the video interpolation performed at main control unit 507 for each of the two or more video streams is identical. For example, parameters defining the characteristics of each display such as the size, scale, magnitude, video display aspect ratio and the like are identical in all displays, and thereby the size, scale, magnitude, video display aspect ratio and the like of the displays are identical when the displays 521, 525 and 520 are played simultaneously.

FIG. 5D discloses a medical imaging system configured to administrate the layout of a video configuration in accordance with another optional layout defined by a fourth video mode, according to FIG. 5A. FIG. 5D shows a medical imaging system 505 comprises a processing unit such as a main control unit 507 operatively connected with a medical imaging endoscope 555 and with a monitor 530 via communication cable or via wireless communication. The operation of the main control unit 507 is disclosed at FIG. 4. The monitor 530 presents a fourth video configuration 534 of a fourth video mode displays three video streams respectively associate with a front camera and two side cameras located at a distal tip 560 of a medical imaging endoscope 555. Fourth video configuration 534 comprises a right video display 521 located at the right side/area of monitor 530, a centric video display 525 located at the center of the monitor 530, and a left video display 520 located at the left side/area of the monitor 530. In such cases, the video display 521, the centric video display 525, and the video display 520 are displayed simultaneously at the monitor 530. In accordance with an embodiment, the three video displays 521, 525 and 520 together provide a panoramic view based on an overlap between field of views of the three cameras, the front camera and the two side cameras. Main control unit 507 may utilize the received three video streams into a single, panoramic video frame, based on an overlap between fields of views of the three cameras.

The fourth video configuration 534 of the fourth video mode may be controlled by control means such as a button/switch set 550 placed within a handle 515 of medical imaging endoscope 555. In some cases, button/knob/switch set 550 may configured with the required buttons and computerized equipment required to send control commands to the medical imaging system 505. Such control commands can comprise instructions to change the layout of the video mode of the video configuration presented on the monitor 530. In some cases, the button/knob set 50 is configured with the required commands to change the layout of the video mode of the video configuration presented on the monitor 530, by utilizing alternative predefined video modes. In some cases, such predefined video modes can comprise data and/or instructions about the size, scale, magnitude, frame rate, video display aspect ratio and additional properties of the video configurations and the locations thereof. In some embodiments, fourth video configuration 534 may be controlled by control means such as buttons, knobs, touch screen, voice control and the like placed within main control unit 507. In some cases, the button/switch set 550 is configured with the required commands to change the layout of the fourth video configuration 534 presented on the monitor 530, by utilizing alternative predefined video modes. In some cases, such predefined video modes can comprise data and/or instructions about the size of the video displays and the locations thereof. In some cases, the sizes of the displays can be different one from each other. In some other cases, the height size of the centric display 525 is at a double size of the right and left video display 521 and 520 respectively. In such an exemplary case, the heights of the centric video display 520, right video display 521, and left video display 530 are equal, yet the width of the right and left video displays, 521 and 520 respectively, can be half of the width of centric video display 525. In another example, the height of the three video displays 521, 520 and 525 are equal, yet the width of the right and left video displays, 521 and 520 respectively, can be one-third of the width of centric video display 525. In yet another example, the height of the three video displays 521, 520 and 525 are equal, yet the width of the right and left video displays, 521 and 520 respectively, can be one quarter of the width of centric video display 525. Persons of ordinary skill in the art should appreciate that the width of the right and left video displays 521 and 520 relatively to the width of centric video display 525 may include other values such as but not limited to 1:1.5, 1:2.5, 1:2.2, 1:3.5, 1:3.7 and more.

In fourth video configuration 534, the centric video display 525 displays the video stream corresponding to the front camera, and the right and left displays 521, 520 display video streams corresponding to the two side cameras of distal tip 560 of medical imaging endoscope 555. In such cases, the fourth video configuration 534 displays the right video display 521, the centric video display 525, and the left video display 520 simultaneously on monitor 530, such that the centers of the three video displays 521, 525 and 520 are maintained at a substantially same level C3, and also, respectively upper edges 521a, 525a, 520a and lower edges 521b, 525b, 520b are at a substantially same level relatively to monitor upper edge 530a and monitor lower edge 530b, respectively.

In some cases, the video interpolation performed at main control unit 507 for each video stream may be either identical or different. For example, parameters defining the characteristics of each display such as the size, scale, magnitude, video display aspect ratio and the like can be different or identical in different displays, and thereby the size, scale, magnitude, video display aspect ratio and the like of the displays may be different one from another. In such exemplary case, the magnitude of the side video displays 521 and 520 can be different from the magnitude of the video display 525 displayed in the center. In some other exemplary cases, the scale, the size, the frame rate, and the like, can be different among the video displays 521, 525 and 520, when the displays 521, 525 and 520 are played simultaneously.

FIG. 5E discloses a medical imaging system configured to administrate the layout of a video configuration in accordance with another optional layout defined by a fifth video mode, according to FIG. 5A. FIG. 5E shows a medical imaging system 505 comprises a processing unit such as a main control unit 507 operatively connected with a medical imaging endoscope 555 and with a monitor 530 via communication cable or via wireless communication. The operation of the main control unit 507 is disclosed at FIG. 4. The monitor 530 presents a fifth video configuration 535 of a fifth video mode that displays three video streams respectively associated with a front camera and two side cameras located at a distal tip 560 of a medical imaging endoscope 555. Fifth video configuration 535 comprises a right video display 521 located at the right side/area of the video configuration, and a left video display 520 located at the left side/area of monitor 530. Fifth video configuration 535 also comprises a centric video display 525 located at the center of monitor 530, below the right video display 521 and the left video display 520. In such cases, the right video display 521, the centric video display 525, and the left video display 520 are displayed simultaneously at the monitor 530 In fifth video configuration 535, the centric video display 525 displays the video stream corresponding to the front camera, and the right and left displays 521, 520 display video streams corresponding to the two side cameras of distal tip 560 of medical imaging endoscope 555.

The fifth video configuration 535 of the fifth video mode may be controlled by control means such as a button/knob/switch set 550 placed within a handle 515 of medical imaging endoscope 555. In some cases, button/knob/switch set 550 may configured with the required buttons and computerized equipment required to send control commands to the medical imaging system 505. Such control commands can comprise instructions to change the layout of the video mode of the video configuration presented on the monitor 530. In some cases, the button/switch set 550 is configured with the required commands to change the layout of the video mode of the video configuration presented on the monitor 530, by utilizing alternative predefined video modes. In some cases, such predefined video modes can comprise data and/or instructions about the size, scale, magnitude, frame rate, video display aspect ratio and additional properties of the video configurations and the locations thereof. In some embodiments, fifth video configuration 535 may be controlled by control means such as buttons, knobs, touch screen, voice control and the like placed within main control unit 507. In some cases, the button/switch set 550 is configured with the required commands to change the layout of the fifth video configuration 535 presented on the monitor 530, by utilizing alternative predefined video modes.

In some cases, the video interpolation performed at main control unit 507 for each video stream of the two or more video streams provided from the center and two side cameras, may be either identical or different. For example, parameters defining the characterizations of each display such as the size, scale, magnitude, video display aspect ratio and the like can be different or equal in different video configurations, and thereby the size, scale, magnitude, video display aspect ratio and the like of the video configurations may be different one from another. In such exemplary case, the magnitude of the side video displays 521 and 520 can be different from the magnitude of the centric video display 525 displayed in the center. In some other exemplary cases, the scale, the size, the frame rate, and the like, can be different among the video displays 521, 525 and 520, wherein the displays 521, 525 and 520 are played simultaneously.

In some cases, the sizes of the video displays can be different one from each other. For example, the height and the width of the right and left video displays 521 and 520 can be one third of the height and the width of centric video display 525. In another example, the height and the width of the right and left video displays 521 and 520 can be half of the height and the width of video display 525. In yet some other examples, the height and the width of the right and left video displays 521 and 520 can be a quarter of the height and the width of video display 525. Persons of ordinary skill in the art should appreciate that the height and the width of the right and left video displays 521 and 520 relatively to the height and the width of centric video display 525 may include other values such as but not limited to 1:1.5, 1:2.5, 1:2.2, 1:3.5, 1:3.7 and more. In such cases, as explained in fifth video configuration 535, the right video display 521, the centric video display 525, and the left video display 520 simultaneously on monitor 530, such that an upper edge 521a of right video display 521 and upper edge 520a of left video display 520 are at a substantially same level relatively to monitor upper edge, and a lower edge 525b of centric video display 525 is at a substantially same level relatively to a monitor lower edge 530b. A lower edge 521b of right video display 521 and lower edge 520b of left video display 520 may be at a substantially same level relatively to an upper edge 525a of centric video display 525 and at a substantially same distance from vertical axis L1. Vertical axis L1 runs along the center of monitor 530 and splits centric video display 525 into to equal parts, in such embodiment, fifth video mode places left edge 521d of right video display 521 and right edge 520c of left video display 520 at the same distance from vertical axis L1.

Persons of ordinary skill in the art should appreciate that the fifth video configuration 535 may be configured to display the right video display 521, the centric video display 525, and the left video display 520 simultaneously on monitor 530, such that lower edge 521b of right video display 521 and lower edge 520b of left video display 520 are at a substantially same level relatively to monitor lower edge 530b, and an upper edge 525a of centric video display 525 is at a substantially same level relatively to a monitor upper edge 530a.

FIG. 6A discloses a medical imaging system configured to administrate the layout of the sixth video configuration comprising more than three displays, in accordance with exemplary embodiment of the disclosed subject. FIG. 6A shows a medical imaging system 605 comprises a processing unit such as a main control unit 607 operatively connected with a medical imaging endoscope 655 and with a monitor 630 via communication cable 640 or via wireless communication. The operation of the main control unit 607 is disclosed at FIG. 4. The monitor 630 can be a display device such as electronic visual display. The medical imaging system 605 is configured to transmit one or more video modes of video data stream and display those video modes simultaneously, wherein the video mode may character each video data stream in different size, scale, magnitude, frame rate, video display aspect ratio, and the like. Monitor 630 presents a sixth video configuration 630 of a sixth video mode that displays three video streams respectively associated with a front camera and two side cameras located at a distal tip 660 of a medical imaging endoscope 655. The main control unit 607 processes the three video streams to display one of the three video streams on more than one video displays, yet parameters defining the characterizations of such more than one video displays, feed from a common video stream, may be different.

Sixth video configuration 631 comprises a right video display 621 typically located at the upper right side/area of monitor 630, a centric video display 625 typically located at the upper center of monitor 630, and a left video display 620 typically located at the upper left side/area of monitor 630. Sixth video configuration 631 displays the right video display 621, the centric video display 625, and the left video display 620 simultaneously at the monitor 630, such that the respectively upper edges 621a, 625a, 620a are at a substantially same level and maintained at a substantially same level relatively to monitor upper edge 630a.

In some cases, the medical imaging system 605 can be configured such that the video content presented at the centric video display 625 may be also displayed at another display of sixth video configuration 631. For example, the medical imaging system 605 can be configured to display the same video content at centric video display 625 and at a larger centric video display 626, wherein sixth video configuration 631 displays lower edge 626b of larger centric video display 626 at a substantially same level relatively to a monitor lower edge 630b. In some cases, the parameters defining the characterizations of the display 625 may be different than the parameters defining the characterizations of the larger centric display 626. For example, parameters such as parameters defining the zoom, the magnitude, the scale, and the like, of display 625 can be different than the parameters of larger centric display 626.

Sixth video configuration 631 of the sixth video mode may be controlled by control means such as a button/knob/switch set 650 placed within a handle 615 of medical imaging endoscope 655. The button/knob/switch set 650 can be configured with the required buttons and computerized equipment required to send control commands to the medical imaging system 605. Such control commands can comprise instructions to change the video mode of the video configuration presented on monitor 630. In some cases, button/knob/switch set 650 is configured with the required commands to change the video mode of the video configuration presented on the monitor 630, by utilizing alternative predefined video modes. In some cases, such video modes can comprise data and/or instructions about the size, scale, magnitude, frame rate, video display aspect ratio and more of the video configurations and the locations thereof. In some embodiments, sixth video configuration 631 may be controlled by control means such as buttons, knobs, touch screen, voice control and the like placed within main control unit 607.

In some cases, the button/knob/switch set 650 may be configured to allow switching between the locations and the parameters defining the characterizations of the video displays. In some embodiments, two side video displays 621 and 620, corresponding to two side cameras, are processed for display such that the right video display 621 corresponds to right side camera is displayed right aligned on the upper right side/area of monitor 630, and left video display 620 corresponds to left side camera is displayed left aligned on the upper left side/area of monitor 630. In one embodiment, the centric video display 625 corresponding to front camera is processed for display such that centric video display 625 is displayed centric of the monitor 530. In one embodiment, user may use button/knob/switch set 650 (placed within handle 615) and or control means (placed within main control unit 607) to display video stream of front camera on video display 625 and on larger centric video display 626, while video stream of right camera is display on right video display 621 and video stream of left camera is displayed on left video display 620. In one embodiment, user may use button/knob/switch/switch set 650 and or control means (placed within main control unit 607) to display video stream of right side/area camera on right video display 621 and on larger centric video display 626, while video stream of front camera is displayed on centric video display 625 and video stream of left camera is displayed on left video display 620. In one embodiment, user may use button/knob/switch set 650 and or control means (placed within main control unit 607) to display video stream of left side camera on video display 620 and on larger centric video display 626, while video stream of front camera is displayed on centric video display 625 and video stream of right camera is displayed on right video display 621.

In some cases, the video interpolation performed at main control unit 607 for each video stream may be either equal or different. For example, parameters defining the characterizations of each display such as the size, scale, magnitude, video display aspect ratio and the like can be different or equal in different displays, and thereby the size, scale, magnitude, video display aspect ratio and the like of the displays may be different one from the other. In such exemplary case, the video interpolation performed on the video displays 621, 626 and 620 is different from the video interpolation performed on the larger centric video display 626.

In some cases, the sizes of the video displays can be different one from each other. For example, the height and the width of the video displays 621, 625 and 620 can be one-third of the height and the width of video display 626. In another example, the height and the width of the video displays 621,625 and 620 can be half of the height and the width of video display 626. In yet some other examples, the height and the width of the video displays 621, 625 and 620 can be a quarter of the height and the width of video display 626. Persons of ordinary skill in the art should appreciate that the height and the width of upper video displays 621, 625 and 620 relatively to the height and the width of lower video display 626 may include other values such as but not limited to 1:1.5, 1:2.5, 1:2.2, 1:3.5, 1:3.7 and more.

In some embodiments of the disclosed subject matter the user operating the medical imaging system 605 may change the video configuration by using predefined video mode saved on a circuit board, such as circuit board 440 of FIG. 4. In some other cases, the user operating the medical imaging system 605 may be able to change one aspect of a displayed video configuration. Thus, the user operating the medical imaging system 605 may be able to change the zoom of the magnitude exclusively in one display, in an ad-hoc fashion. For example, the user operating the medical imaging system 605 may utilize the button/knob/switch set 650 and or the control means (placed within main control unit 607) to unexpectedly change the zoom or different magnitude of video displays 621, 625, 620 and 626 without changing other aspects of the characterizations of the displays. For example, in some cases, the zoom change may be only on video display 625 or on video display 626.

In some embodiments of the disclosed subject matter, the button/knob/switch set 650 and or the control means (placed within main control unit 607) may be configured to allow user operating the medical imaging system 605 to change the location of each display on monitor 630. For example, in case right video display 621 displays video content stream captured and streamed by a first side camera (not shown), and the left video display 620 displays video content captured and streamed by a second side camera (not shown), and the centric video display 625 displays video content captured and streamed by a front camera (not shown), the user utilizing the button/knob/switch set 650 may be able to change the displays locations of the video displays 621, 620, 625 and 626 on monitor 630. Such a change of the display locations may cause to left video stream to be seen in the right side/area of video configuration 631, right to the centric video display 625 and to left video stream to be seen at the right side/area of video configuration 631 right to centric video display 625. In some cases, the button/knob/switch set 650 and or the control means (placed within main control unit 607) may be configured also to allow user operating the medical imaging system 605 to change the parameters defining the characterizations of the displays. For example, in case displays video content captured and streamed by the front camera (not shown), the button/knob/switch set 650 and or the control means (placed within main control unit 607) can be configured to allow changing the location of centric video stream and the parameters defining the characterizations of the display such that centric video stream can be seen at left side/area of monitor 630 (on left side/area video display 621) instead of the left video stream and the left video stream to be seen at the center of monitor 630 (on centric video display 625) instead of left video stream. In such cases, button/knob/switch set 650 and or the control means (placed within main control unit 607) may also configure to simultaneously display one of the three video streams to be also displayed on larger centric video display 626. Larger centric video display 626 typically located at the center of the lower part of monitor 630 and may be characterized by a larger size compared to the video displays 621, 625, 620 typically located at the upper part of monitor 630.

Persons of ordinary skill in the art should appreciate that sixth video configuration 631, may configured to display the right video display 621, the centric video display 625, the left video display 620 and larger centric video display 626 simultaneously on monitor 630, such that lower edges 621b, 625b and 620b of right video display 621, centric video display 625 and left video display 520, respectively are at a substantially same level relatively to monitor lower edge 630b, and an upper edge 626a of larger centric video display 626 is at a substantially same level relatively to a monitor upper edge 630a.

In some embodiments, video displays 621, 625, 620 are processed for display such that right edge 621c of right video display 621 and right edge 626c of larger centric video display 626 are maintained at a substantially same vertical axis and also, left edge 620d of left video display 620 and left edge 626d of larger centric video display 626 are maintained at a substantially same vertical axis. FIG. 6B discloses a medical imaging system having a non-flat monitor and configured to administrate the layout of the seventh video configuration comprising more than three displays, according to FIG. 6A. A medical imaging system 605 comprises a processing unit such as a main control unit 607 operatively connects with a medical imaging endoscope 655 and with a non-flat monitor 630′ via communication cable 640 or via wireless communication. The communication cable 640 can be configured and designed to transmit video displays according to diverse video signaling formats, as aforementioned. In one embodiment, monitor 630′ can be a flexible display device. In some cases, the flexible display device also known as an electronic visual display, can be based on technology such as flexible electronic paper, flexible OLED-based displays, and the like.

The monitor 630′ presents a seventh video configuration 632 of a seventh video mode displays three video streams respectively associate with a front camera and two side cameras located at a distal tip 660 of a medical imaging endoscope 655 and comprising a right video display 620 typically located at the upper right side/area of monitor 630′, a centric video display 625 typically located up at the center of the monitor 630′, and a left video display 621 typically located at the upper left side/area of the monitor 630′. Seventh video configuration 632 displays the right video display 621, the centric video display 625, and the left video display 620 simultaneously at the monitor 630′, such that the respectively upper edges 621a, 625a, 620a are at a substantially same level and maintained at a substantially same level relatively to monitor upper edge 630′a.

In some cases, the video interpolation performed at main control unit 607 for each video stream may be either equal or different. For example, parameters defining the characterizations of each display such as the size, scale, magnitude, video display aspect ratio and the like can be different or equal in different displays, and thereby the size, scale, magnitude, video display aspect ratio and the like of the displays may be different one from the other. In such exemplary case, the video interpolation performed on the video displays 621, 626 and 620 is different from the video interpolation performed on the larger centric video display 626.

In some cases, the medical imaging system 605 can be configured such that, the video content presented at the centric video display 625 may also be displayed at another display of seventh video configuration 632. For example, the medical imaging system 605 can be configured to display the same video content at centric video display 625 and at a larger centric video display 626, wherein seventh video configuration 632 displays lower edge 626b of larger centric video display 626 at a substantially same level relatively to a monitor lower edge 630′b. In some cases, the parameters defining the characterizations of display 625 may be different than the parameters defining the characterizations of larger centric display 626. For example, parameters such as parameters defining the zoom, the magnitude, the scale, and the like, of video display 625 can be different than the parameters of larger centric display 626. In some embodiments, video displays 621, 625, 620 are processed for display such that right edge 621c of right video display 621 and right edge 626c of larger centric video display 626 are maintained at a substantially same vertical axis and also, left edge 620d of left video display 620 and left edge 626d of larger centric video display 626 are maintained at a substantially same vertical axis.

In some cases, the sizes of the video displays can be different one from the other. For example, the height and the width of the video displays 621, 625 and 620 can be one-third of the height and the width of video display 626. In another example, the height and the width of the video displays 621,625 and 620 can be half of the height and the width of video display 626. In yet some other examples, the height and the width of the video displays 621, 625 and 620 can be a quarter of the height and the width of video display 626. Persons of ordinary skill in the art should appreciate that the height and the width of video displays 621, 625 and 620 relatively to the height and the width of video display 626 may include other values such as but not limited to 1:1.5, 1:2.5, 1:2.2, 1:3.5, 1:3.7 and more.

In some embodiments of the disclosed subject matter, the user operating the medical imaging system 605 may change the video configuration by using predefined video modes saved on a circuit board, such as circuit board 440 of FIG. 4. In some embodiments of the disclosed subject matter, control means such as a button/knob/switch set 650 and or control means (placed within main control unit 607) may be configured to allow user operating the medical imaging system 605 to change the location of each display on monitor 630′ as describes in FIG. 6A.

In accordance with an embodiment, the three video displays 621, 625 and 620 together provide a panoramic view based on an overlap between field of views of the three cameras, the front camera and the two side cameras. Main control unit 607 may utilize the received three video streams into (1) a single, panoramic video frame, based on an overlap between field of views of the three cameras and display on upper part of monitor 630, and (2) larger centric video display 626 adapted to display one of the three video streams, according to a user selection.

Persons of ordinary skill in the art should appreciate that seventh video configuration 632, may configured to display the right video display 621, the centric video display 625, the left video display 620 and larger centric video display 626 simultaneously on monitor 630′, such that lower edges 621b, 625b and 620b of right video display 621, centric video display 625 and left video display 520, respectively are at a substantially same level relatively to monitor lower edge 630′b, and an upper edge 626a of larger centric video display 626 is at a substantially same level relatively to a monitor upper edge 630′a.

In another embodiments (not shown), video configuration according to FIGS. 6A-6B, may configured to display on one monitor display on display, such right video display 621, centric video display 625, left video display 620 and larger centric video display 626 simultaneously on monitor 630, 630′, while right video display 621, centric video display 625, left video display 620 are overlayed larger centric video display 626 without transparency, leave other settings as they were set by the video configuration. The positioning of right video display 621, centric video display 625, left video display 620 may be on top of larger centric video display 626 such, upper edges 621a, 625a and 620a of right video display 621, centric video display 625 and left video display 520, respectively are at a substantially same level relatively to upper edge 626a of larger centric video display 626. The positioning of right video display 621, centric video display 625, left video display 620 may be on button of larger centric video display 626 such, lower edges 621b, 625b and 620b of right video display 621, centric video display 625 and left video display 520, respectively are at a substantially same level relatively to lower edge 626b of larger centric video display 626.

In the above embodiments, wherein monitor 630, 630′ displays the displays on display embodiment, a user of the medical imaging system 605 may switch between each of the video data streams to be display on the larger centric video display 626, and moreover the user may choose with the control means to display one, two or three of the video data streams on the larger centric display 626.

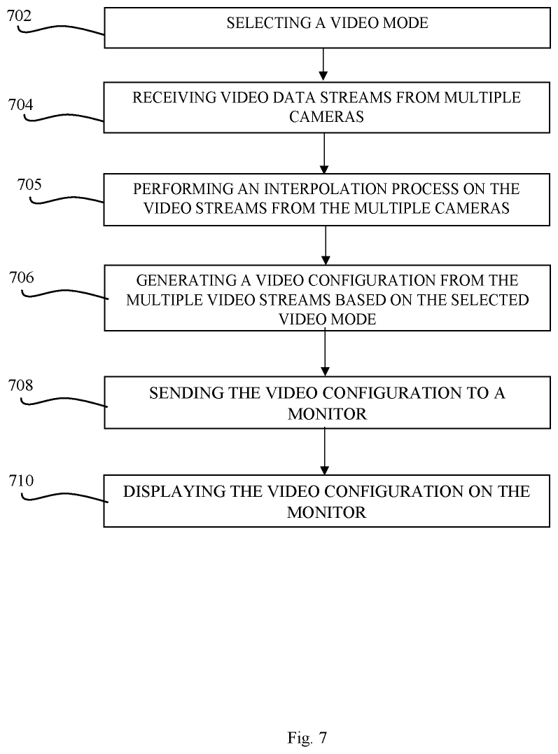

FIG. 7 is a flow chart illustrating the steps of displaying a selected video configuration of a selected video mode on a monitor of a medical imaging system, in accordance with embodiments shown in FIGS. 5-6.

At step 702, a medical imaging system receives a video mode defining a video layout of the two or more data streams captured by two or more cameras, for example multiple cameras, of the medical imaging system. The video mode may be received via control means operated by a user of the medical imaging system.

At step 704, a processing unit, such as main control unit, receives two or more video data streams of video content captured by the two or more cameras located at a distal tip of a medical imaging endoscope.

At step 705, the medical imaging system performs a video interpolation process on the two or more video data streams based on the selected video mode. The video interpolation process performed on the two or more video data streams may be either identical or different. For example, video configurations to be displayed on a monitor dictate that at least a portion of the parameters defining the video characteristics of each video data stream be different for each of the video streams. Such parameters may include size, scale, magnitude, video display aspect ratio and the like can. The video characteristics of the video interpolation process define the manner of displaying each video data stream on the monitor. For example, in embodiments the main control unit receives three video data streams, the interpolation process may be identical for streams #1 and #3 and have a different aspect ratio for stream #2, based on video characteristics stored in a memory module of the device.

At step 706, the medical imaging system generates a video configuration based on the two or more video data streams, and according to video characteristics defined by video mode. Such video characteristics may comprise at least one of size, scale, magnitude, frame rate and video display aspect ratio.

At step 708, the medical imaging system transmits the video mode to the monitor, and at step 710, the medical imaging system displays the video configuration layout defined by the video mode, said video configuration comprises video data streams of at least two of the two or more video data streams displayed simultaneously.

In another embodiment, the subject matter discloses a method for receiving three video streams from three cameras of the medical imaging system. One of the video configurations enabled to the user of the medical imaging system defines a layout having four displays to be simultaneously displayed on the monitor. Three of the four displays are provided from the three cameras, and have identical video characteristics. The fourth video display comprises a video data stream provided from one of the three cameras, but has a video characteristic different from the other three video displays.

In some embodiments, a visual sign/indicator may be used to indicate which of the video streams, respectively, is shown on which video display, in each of the video configurations mentioned above. Such visual sign/indicator may be added to each video stream during the processing/utilizing of the video streams within a main control unit. The operation of the main control unit 507, 607 is disclosed at FIG. 4. One of ordinary skill in the art would appreciate that any type of indication, such as “Left camera stream”, “Right camera stream”, “Centric camera stream” may be used to single out the video source display on each video displays. In one embodiment, letters “L”, “C”, “R” are used for indication and/or emphasizing—L for left camera, R for right camera, C for centric camera. The visual sign/indicator may appear on each video display in such a way that the sign/indicator is not interrupting the user of seeing the video streams, for example and not limited to in proximity to video display edges, on the upper right corner of the video display, on the upper left corner of the video display, on the lower right corner of the video display, on the lower left corner of the video display. The user may choose to change the location and types of signal/indicator by using a button/knob set and or control means. The operation of the button/knob set and or control means is disclosed at FIGS. 5-6. In one embodiment, the signal/indicator may appear on the same place on each video display. In one embodiment, the signal/indicator may appear on different places on each video display.

1. A medical imaging system for generating a video configuration from at least two cameras of a medical imaging endoscope, wherein the medical imaging system comprises a processing unit, wherein the processing unit is operatively connected to the medical imaging endoscope and to a monitor, and wherein the processing unit of the medical imaging system is instructed to:
receive at least two video data streams captured from the at least two cameras located at a distal tip of the medical imaging endoscope;
receive from the medical imaging system a video mode defining a layout of the at least two video data streams;
perform a video interpolation on each of the at least two video data streams according to the video mode;
generate a video configuration for the at least two video data streams, wherein the video configuration characters each video data stream of the at least two video data streams in different size, scale, magnitude, frame rate and video display aspect ratio in accordance with the received video mode;
send the video configuration to the monitor, and
display the video configuration layout defined by the video mode of the at least two video data streams simultaneously on the monitor.

2. The medical imaging system of claim 1, wherein the medical imaging system further comprising a control means configured to receive the video mode that dictates the layout of the video configuration.

3. The medical imaging system of claim 2, is further designed to modify the parameters defining the characterizations of the displays of the at least two video data streams for changing the layout of a displayed video configuration.

4. The medical imaging system of claim 2, wherein the video interpolation is performed independently on each of video data stream of the at least two video data streams.

5. The medical imaging system of claim 2, wherein the video interpolation comprising utilizing parameters which define characteristics of the display, the parameters are selected from a list comprising a number of video frames to output, frame rate, timestamps assuming constant frame rate, video display aspect ratio, pixel format defining the memory layout of each pixel in a bitmap, overlap lines and magnitudes, such that the processing unit is further configured to receive data of the at least two video data streams, follow by perform the video interpolation according to a predefined video mode for obtaining the video configurations.

6. The medical imaging system of claim 2, wherein the video display aspect ratio is 4:3, 16:9, 16:10, 32:9.

7. A method for displaying a video configuration of at least two cameras of a medical imaging endoscope by a medical imaging system comprises a processing unit operatively connected with the medical imaging endoscope and with a monitor, the method comprising:
receiving at least two video data streams captured by at least two cameras located at the medical imaging endoscope in the processing unit;
receiving from the medical imaging system a video mode defining a layout of the at least two video data streams;
performing a video interpolation on each of the at least two video data streams based on the received video mode;
generating a video configuration from the at least two video data streams, wherein the video configuration defines each video data stream of the at least two video data streams is different in one or more video characteristics;
transmitting the video configuration to the monitor, and
displaying the video configuration layout on the monitor, such that the at least two video data streams are displayed simultaneously.

8. The method of claim 7, wherein the video mode is received from control means of the medical imaging system.

9. The method of claim 8, wherein the control means comprise at least one of buttons, switches, knobs placed within a handle of the medical imaging endoscope.

10. The method of claim 8, wherein the control means comprise at least one of buttons, switches, knobs placed within the processing unit.

11. A method for displaying a layout of a video configuration based on video data streams captured by three cameras of a medical imaging device, comprising:
receiving three video data streams captured by the three cameras located within the medical imaging device;
generating the video configuration from the three video data streams using a main control unit of the medical imaging device and based on a predefined video mode, said video configuration comprises four video displays, wherein three video displays of the four video displays relates to the video data streams captured by the three cameras and a fourth video display comprises a video data stream captured by a selected camera of the three cameras, wherein the four video displays are simultaneously displayed on a monitor.

12. The method of claim 11, wherein the fourth video display has a video characteristic different from the three video displays' video characteristic.

13. The method of claim 12, wherein the video characteristic is size.

14. The method of claim 12, wherein the video characteristic is scale.

15. The method of claim 12, wherein the video characteristic is magnitude.

16. The method of claim 12, wherein the video characteristic is frame rate.

17. The method of claim 12, wherein the video characteristic is video display aspect ratio.

18. A method for displaying a video configuration based on video streams captured by three cameras of a medical imaging endoscope by a medical imaging system comprises a processing unit operatively connected with the medical imaging endoscope and with a monitor, the method comprising:
receiving three video data streams captured by the three cameras located within the medical imaging endoscope in the processing unit;
receiving from the medical imaging system a video mode defining a layout of the three video data streams into four video displays, to be simultaneously displayed on the monitor;
performing a video interpolation on each of the three video data streams based on the video mode;
generating a video configuration based on the three video data streams, wherein the video configuration comprises three video displays having different video characteristics, each display of the three displays is provided from a different video stream of the characters each video data stream in different size, scale, magnitude, frame rate and video display aspect ratio and wherein the video mode comprises display of the three video data streams at the four video displays;
setting parameters defining the characterizations of each of the four video displays of the three video data streams in accordance with the received video configuration;
transmitting the video configuration to the monitor, and
displaying the video configuration layout defined by the video mode of the three video data streams on four video displays simultaneously.

19. A medical imaging system comprising:
a monitor, and
a medical imaging endoscope comprising three cameras adapted to capture a front video stream and two side video streams, and a handle,
wherein the medical imaging system comprises a control means configured to receive commands from a user of the medical imaging system and send control commands to a processing unit,
wherein the processing unit is operatively connected with the medical imaging endoscope and the monitor,
wherein the processing unit comprises a circuitry board configured to perform a video interpolation on each video stream of the front video stream and the two side video streams based on a video mode in order to display a video configuration on the monitor,
wherein the video configuration comprises three video displays concurrently to provide a panoramic view based on an overlap between field of views of the three cameras,
wherein the processing unit is utilized to receive the front video stream and the two side video streams into a single, panoramic video frame, based on an overlap between field of views of the three cameras;
wherein the video configuration comprises and display on a first area of the monitor a larger centric video display and display on a second area of the monitor one of the three video streams, according to the video mode.

20. The medical imaging system of claim 19, wherein the first area of the monitor is an upper area of the monitor and the second area of the monitor is a lower area of the monitor.

21. The medical imaging system of claim 19, wherein the first area of the monitor is a lower part of the monitor and the second area of the monitor is an upper part of the monitor.
